(19) United States
(12) Patent Application Publication
(10) Pub. No.: US 2003/0165533 A1
(43) Pub. Date: Sep. 4, 2003

(54) Chinese traditional medicines for psoriasis

(76) Inventors: Hsu-Shan Huang, Taipei (TW); Rong Fu Chen, Taipei (TW)

Correspondence Address: DYKAS, SHAVER & NIPPER, LLP, P O BOX 877, BOISE, ID, 83701-0877, US

(21) Appl. No.: 10/350,408

(22) Filed: Jan. 24, 2003

Related U.S. Application Data
(62) Division of application No. 10/050,060, filed on Jan. 14, 2002.

(51) Int. Cl. A61K 35/78

(52) U.S. Cl. 424/195.18; 424/740; 424/746; 424/748; 424/773; 424/736; 424/778; 424/758

(57) ABSTRACT

The invention relates to disclose some Chinese formula for psoriasis. Whether treatment for psoriasis is targeted at both the hyperproliferative and inflammatory aspect of the topical therapy. Specially, the administration dosage is soft gel, cream, tincture and aerosol etc. topical dosages.

### PRIORITY

This application is a Divisional of the utility application filed on Jan. 14, 2003 entitled Chinese traditional medicines for psoriasis with Ser. No. 10/050,060.

### DESCRIPTION

### BACKGROUND OF THE INVENTION

Field of the Invention

The invention relates to disclose some Chinese formula for psoriasis. Whether treatment for psoriasis is targeted at both the hyperproliferative and inflammatory aspect of the topical therapy.

Psoriasis is a widespread, inflammatory and scaling skin disease, which is characterized by abnormal keratinocyte proliferation and differentiation of the epidermis, accumulation of polymorphonuclear leukocytes in the skin. Dominant and interdependent features of psoriasis are epidermal hyperproliferation, disturbed keratinocyte differentiation, and inflammation of the dermis and epidermis.

However, the etiology of this very distressing skin disorder is unknown. The treatment for psoriasis is targeted at both the hyperproliferative and inflammatory aspect of the topical therapy. Müller K, et al., J Med Chem. 39, 3132-8,1996 has reported some dihydroxy-9(10H)-anthracenones that have inhibited keratinocyte growth, 5-lipoxygenase, and the formation.

Current treatment for psoriasis may be topical or systemic. Müller K., Curr. Pharm. Design, 6, 901-18, 2000 has reported indications for systemic treatment are failure to respond to topical treatment and severe or life-treatening forms of psoriasis.

Müller K and Huang H S., Chin Pharm J. 48, 337-54, 1996 have suggested the biological activity of drugs useful as antipsoriatic agents may be evaluated by their antiproliferative activity in cell cultures and antioxidative activity in vitro, since there is no appropriate animal model of psoriasis.

CTMs (Chinese traditional medicines) have been extensively used to treat psoriasis and have acquired considerable favor among many of the patients. The earliest description of psoriasis in ancient Chinese medical literature may be found in Sui-Dynasty, 581-618 AD. According to Chinese literatures “Bian Zheng Shi Zhi”, Lin X R. J. Dermato. 20, 746-55, 1993 collected a number of clinical psoriasis cases report, and distinguished psoriasis into 2 or 3 types, namely “Blood-Heat” type and “Blood deficiency-Dryness” type or “Blood-Heat”, “Blood deficiency-Dryness” and “Blood stasis” type. While in also described prescription (I) to treat “Blood-Heat” type, that including Caulis Spatholobi 2, Radix Rehmanniae Exsiccata 6, Radix Lithospermi 13, Radix Salviae Miltiorrhizae 14, Radix Paeoniae Veitchii 17, Rhizoma Imperatae 23, and Flos Sophorae 27. Prescription (II) to treat “Blood deficiency-Dryness” type that including Caulis Spatholobi 2, Radix Angelicae Sinensis 3, Rhizoma Smilacis Glabrae 4, Radix Rehmanniae Exsiccata 6, Radix Ophiopogonis Japonici 7, Radix Asparagi 8, Nidus Vaspae 10, and Radix Salviae Miltiorrhizae 14. Prescription (III) to treat “Blood stasis” type that including Canlis Spatholob 2, Rhizoma Sparganii 5, Herba Hedyotis 11, Rhizoma Curcumae Aeruginosae 15, Pericarpium Citri Reticulatae 18, Flos Carthami 19, Semen Amygdalus Persicae 20, and Ramulus Euonymi 26.

It is well know that CTMs are compound medicines, which are combined with various crude drugs, and the exhibition of their pharmacologic activity is the result of the pharmacodynamic interaction of the combined drugs and their components. Zhonghua Bencao have state Radix Tripterygiim Wilfordii 1 is the plant of Tripterygium wilfordii Hook. f., have containing some alkaloids, diterpene, triterpene ingredients, such as Wilfordine, Tripterin, Triptolide, Tripdiolide, Triptonide, Celastrol, Tripterifordin, Triptofordin A-G, Triptogelin A₁₋₄, Triptogelin B₁, Triptogelin C₁ and C₄, Triptogelin D₁₋₂,Triptogelin E₁₋₄, Triptogelin G₁, Triptoquinone A-G, Triptonoterpen, Neotriptonodiol, Triptonodiol, Neotriptonolide.

Caulis Spatholobi 2 is the plant of Spatholobus suberectus Dunn have containing some Fridelan-3β-ol, daucosterol, β-sitosterol, 7-oxo-β-sitosterol, formononetin, ononin, prunetin, 9-methoxycoumestrol, medicago, afromosin, 7-dihydroxy-6-methoxy-dihydroflavonol, chalcone, protocatechuic acid, epicatechin, licochalcone A, isoliquiritigenin, 5α-stigmastane-3β,6α-diol, 2′,4′,3,4-tetrahydroxy daidzein, stigmast-5-ene-3β,7α-diol, cajanin.

Radix Angelicae Sinensis 3 is the plant of Angelicae sinensis (Oliv.) Diels have containing essential oil, nicotinic acid, butylidene phthalide, n-Valerophonone-O-carboxylic acid, carvacrol, phenol, ocresol, p-cresol, guaiacol, 2,3-dimethylphenol p-ethylphenol, isoeugenol, m-ethylphenol, 4-ethylresorcinol, 2,4-dihydroxy-acetophenone, vanillin, ligustilide, adenine, myrcene, α-pinene, β-ocimine-X, alloocimine, bicycloelemene, cadinene, 6-n-butyl-1,4-cycloheptdiene, 2-methyldodecan-5-one, copaene, 1,5-trimethyl-2-formyl cyclohexa-2,5-diene-4-one, acoradiene, uracil, acetophenone, β-bisabolene, isoacoradiene, bergamotene, brefeldin, eucarvonel, threonine, senkyunolide, 3,4-dimethyl-benzaldehyde, trans-β-famesene, γ-elemene, n-butylphthalide, n-butylidenephthalide, safrole, leucine, camphoric acid, azelaic acid, sebacic acid, myristic acid, phthalic 6-methoxy-7-hydroxycoumarin, anhydride, α-cedrene, vanillic acid, p-ethyl-benzaldehyde, verbenone, 2,4,6-trimethyl benzaldehyde, angelicide, β-selinene, 1-tetradecanol, β-sitosterol, daucosterol, palmitic acid, succinic acid. ferulic acid, cuparene.

Rhizoma Smilacis Glabrae 4 is the plant of Smilax glabra Roxb. have containing tannin, saponin, resin, astilbin, engeletin, 3-O-caffeoylshikimic acid, shikimic acid, ferulic acid, β-sitosterol, quercetin, kaempferol.

Rhizoma Sparganii 5 is the plant of Sparganium stoloniferum Buch. -Ham. have containing benzeneethanol, dehydrocostuslactone, hexadecanoic acid, 1,4-benzenediol, β-elemene, 2-furanmethanol, 2-acetylpyrrole, stigmasterol, 1-hydroxy-2-acetyl-4-methylbenzene, 9-octadecenoic acid, 3-phenyl-2-propenoic acid, formonetin, β-sitosterol, 11-eicosenoic acid, decanedioic acid, benzoic acid, azelaic acid, 3,4-dihydro-8-hydroxy-3-methyl-1H-2-benzopyran-4-one, daucosterol, succinic acid, sanleng acid, 9,11-octadecadienoic acid, 9,12-octadecadienoic acid, 9-hexadecenoic acid, 19-nonadecenoic acid.

Radix Rehmanniae Exsiccata 6 is the plant of Rehmannia glutinosa (Gaertn.) Libosch. Ex Fisch. et Mey. have containing Leonuride, rehmannoside A-D, ajugol, aucubin, melittoside, rehmaglutin A-D, acteoside, isoacteoside, monometittosid, geniposide, ajugoside, 6-O-E-feruloylajugol, jioglutin D-E, jioglutoside A, jioglutolide, 6,8-dihydroxyboschnialactone, echinacoside, cataepolgenin, grardoside, Mioporosidegenin, rehmaionoside A-C, rehmapicroside, purpureaside C, Jionoside A1, Jionoside B1, cistanoside A, cistanoside F, glutinoside, some organic acid.

Radix Ophiopogonis Japonici 7 is the plant of Ophiopogon japonicus (L. f.) Ker-Gawl. have containing linalool, ruscogenin, ophiopogonin B, ophiopogonin D, jasmololone, (23S,24S,25S)-23, 24-dihydroxyruscogenin, methylophiopogonanone A-B, ophiopogone A, ruscogenin-1-O-sulfate, terpinen-4-ol, glycerol, longifolene, cyperene, guaiol,

Radix Asparagi 8 is the plant of Asparagus cochinchinensis (Lour.) Merr. have containing methylprotodioscin, pseudoprotodioscin, yamogenin, diosgenin, sarsasapogenin, asparagus polysaccharide A-D, oligosaccharide I-VII, and smilagenin.

Olibanum 9 is the plant of Boswellia carterii Birdw have containing α,β-boswemc acid, olibanoresene, O-acetyl-β-boswellic acid, thujone, dihydroroburic acid, epilupeol acetate, tirucallol, pinene, Arabic acid, 5-hydroxy-p-menth-6-en-2-one, bassorin, myrtenic acid, limonene, 10-hydroxy-4-cadinen-3-one, myrtenal, phellandral, α,β-phellandrene, pinocamphone, carvotanacetone, cuminaldchyde, 1-acetyl-4-isopropenylcyclopentene, 3,6,6-trimethylnorpinan-2-one, nopinone, verbenone, α-amyrenone, isopropylidenecyclohexane, piperitone, cryptone, carvone, α-ampholenaldehyde, p-menth-4-en-3-one, O-methylacetophenone, perilla-aldehyde, eucarvone, 2,4-dimethylacetophenone, γ-campholenaldehyde, 11-keto-α-amyrenone.

Nidus Vaspae 10 is the animal of Polistes mandarinus Saussure have containing resin, wax, saccharide.

Herba Hedyotis 11 is the plant of Hedyotis Diffusa Willd. have containing asperuloside, hentriacontane, asperulosidic acid, β-sitosterol, deacetylasperulosidic acid, scandoside, geniposidic acid, stigmasterol, 5-O-p-hydroxycinnamoyl scandoside methylester, ursolic acid, p-coumaric acid, β-sitosterol-β-D-glucoside, 2-methyl-3-hydroxy-4-methoxyanthraquinone, oleanolic acid, scandoside methylester, 2-methyl-3-hydroxyanthraquinone, 2-methyl-3-methoxyanthraquinone, 5-O-feruoyl scandoside methylester, 5-O-p-methoxy cinnamoyl scandoside methylester.

Indigo Naturalis 12 is the plant of Baphicacanthus cusia (Nees) Bremek., Polygonum tinctorium Ait., Indigofera tinctoria L., and Isatis indigotica Fort. have containing indirubin, indigo.

Radix Lithospermi 13 is the plant of Lithospermum erythrorhizon Sieb. et Zucc. have containing Shikonin, deoxyshikonin, 1-eicosanol, isovalerylshikonin, 1-tetracosanol, caffeic acid, isobutyrylshikonin, 1-docosanol, stearyl alcohol, α-methyl-n-butyrylshikonin, β,β-dimethylacrylshikonin, lithospermidin A, lithospermidin B, β-hydroxyisobutyrylshikonin,

Radix Salviae Miltiorrhizae 14 is the plant of Salviae miltiorrhiza Bge. have containing cryptotanshinone, diterpen, nortanshinone, tanshinone I, IIA-IIB, V-VI, stigmasterol, Isotanshinone I, IIA-IIB, neocryptotanshinone, Δ¹-dehydrotanshinone IIA, isocryptotanshinone, 1-ketoisocryptotanshinone, hydroxytanshinone IIA, dihydrotanshinone, methyl tanshinonate, dihydroisotanshinone I, formyltanshinone, methylenedihydrotanshinone, 1,2,5,6-tetrahydrotanshinone I, β-sitosterol, methylene tanshiquinone, dihydrotanshinquinone, danshexinkum A-D, tanshindiol A-C, miltirone, Δ¹-dehydromiltirone, 4-methylenemiltirone, miltionone I-II, salvilenone, tanshinlactone, dihydrotanshinlactone, ursolic acid, danshenspiroketallactone, epidanshenspiroketallactone, cryptoacetalide, epicryptoacetalide, salvinone, salviolone, miltiodiol, miltipolone, norsalvioxide, ferruginol, saiviol, sugiol, salvianic acid A-C, D(+)-β-(3,4-dihydroxyphenyl) lactic acid, salvianolic acid, rosmarinic acid, methyl rosmarinate, monomethyl lithospermate, dimethyl lithospermate, ethyl lithospermate, lithospermic acid B, protocate chualdehyde, isoferulic acid, baicalin, 5-(3-hydroxypropyl)-7-methoxy-2-(3′-methoxy-4′-hydroxy-phenyl)-3-benzo[b]furancarbalde hyde, daucosterol, tigogenin, isoimperatorin.

Rhizoma Curcumae Aeruginosae 15 is the plant of Curcumae aeruginosae Roxb. have containing essential oil, sesquiterpenoid such as, curzerenone, borneol, germacrone, pinene, curcumene, camphene, limonene, 1,8-cineole, turmerone, terpinene, isoborneol, caryophyllene, caryophyllene epoxide, curcurmenol, arturmerone, curdione, aerugidiol, difurocumenone, isocurcumenol, curcuminoids

Myrrha 16 is the plant of Commiphora myrrha Engl. have containing heerabomyrrholic acid, commiphoric acid, commiphorinic acid, heerabomyrrhol, heeraboresene, commiferin, eugenol, m-cresol, pinene, limonene, cuminaldehyde, cinnamic aldehyde, heerabolene, 8α-methoxyfuranodiene, 8α-acetylfuranodiene, curzerene, lindestrene, furanoeudesma-1,3-diene, furanodiene, resin, gum,

Radix Paeoniae Veitchii 17 is the plant of Paeonia veitchii Lynch have containing paeoniflorin, benzoyl paeoniflorin,

Pericarpium Citri Reticulatae 18 is the plant of Citrus reticulata Blanco have containing carvacrol, α-farnesene, citronellal, (1,1-dimethylethy)-benzenemethanol, terpinolene, sabinene hydrate, 5,7,4′-trimethoxyflavone, ferulic acid, α-terpineol, decanal, 5,7,8,3′,4′-pentamethoxyflavone, 5,7,8,4′-tetramethoxyflavone, α-terpinene, 5-O-desmethylcitromitin, 5-hydroxy-7,8,4′-trimethoxyflavone, sinensetin, α-thujene, sabinene, 5,4′-dihydroxy-7,8-dimethoxyflavone, nobiletin, octanal, pinene, 4-terpineol, 5,6,7.3′,4′-pentamethoxyflavone, benzyl alcohol, citromitin, α-ocimene, p-cymene, 5,6,7,8,3′,4′-hexamethoxyflavone, perillaldehyde, xanthomicrol, limonin, limonene, 5-hydroxy-6,7,3′,4′-tetramethoxyflavone, neral, thymol, p-sitosterol, octanol, 5-hydroxy-6,7,8,3′,4′-pentamethoxyflavone, citronellol, α-phellandrene, β-myrcene, 5,7,4′-trihydroxy-6,8,3′-trimethoxyflavone, linalool, sudachiflavone, neohesperidin, 5-hydroxy-6,7,8,4′-tetraroethoxyflavone, tangeritin, 3,7-dimethyl-7-octenal, nerol, 4′-hydroxy-5,6,7,8-tetramethoxyflavone, 5,5′-oxydimethylene-bis(2-furaldehyde), 5,6,7,8,4′-pentamethoxyflavone, γ-terpinene, 5,4′-dihydroxy-6,7,8-trimethoxyflavone, hesperidin.

Flos Carthami 19 is the plant of Carthamus tinclorius L. have containing carthamin, precarthamin, safflor yellow A-B, safflomin A, chlorogenic acid, caffeic acid, catechol, pyrocatechol, dopa, and volatility compounds

Semen Amygdalus Persicae 20 is the plant of Amygdalus persica L. have containing citrostadienol, β-sitosterol, β-sitosterol-3-O-β-D-glucopyranoside, 7-dehydroavenasterol, 24-methylene cycloartanol, 3-feruloylquinic acid, β-sitosterol 3-O-β-D-(6-O-palmityl)glucopyranoside, campesterol-3-O-β-D-glucopyranoside, Chlorogenic acic, β-sitosterol-3-O-β-D-(6-O-oleyl)glucopyranoside, triolein, campesterol, tryptophane, campesterol-3-O-β-D-(6-O-oleyl)glucopyranoside, prunasin, campesterol-3-O-β-D-(6-O-palmityl)glucopyranoside, oleic acid, 3-caffeoxyquinic acid, methyl-α-D-fructofuranoside, methyl-β-D-glucopyranoside, lineleic acid, amygdalm.

Radix Angelicae Biserratae 21 is the plant of Angelicae biserrata (Shan et Yuan) Yuan et Shan, have containing columbianetin, columbianetin acetate, anpubesol, osthol, isoimperation bergapten, xanthotoxin, thymol, columbianadin, p-cymene, angelol D, columbianetin-β-D-glucopyranoside, pcresol, γ-aminobutyric acid, β-cedrene, oxocyclohexandecan-2-one, α-pinene, humulene, dodccylisopropylether, eremophilene, 4,4′-methylenebis(2,3,5,6-tetramethyl)phenol, nerolidol, α-cedrene, α-longipinene, sylvestrene, 3-methylnonane, aphelladrene.

Radix Angelicae Hangbaizhi, or Radix Angelicae Qibaizhi 22 is the plant of Angelicae dahuricae and some variation spp. (Umbelliferae) have containing some lactones, such as imperatorin, isoimperatorin, alloisoimperatorin, oxypeucedanin, isooxypeucedanin, oxypeucedanin hydrate, byakangelicin, byakangelicol, neobyakangelicol, pabulenol, sitosterol, phellopterin, xanthotoxol, bergapten, 5-methoxy-8-hydroxypsoralen, palmitic acid,

Rhizoma Imperatae 23 is the plant of Imperata cylindrical (L.) Beauv. var. major (Nees) C. E. Hubb. have containing arundoin, cylindrin, isoarborinol methyl ether, fernenol, isoarborinol, and arborinol methyl ether.

Herba Artemisiae Anomalae 24 is the plant of Artemisia anomala S. have containing arteanoflavone, coumarin, eupatilin, tricin, herniarin, scopoletin, simiarenol, umbelliferone, salvigenin, reynosin, armexifolin, anomalamide, palmitic acid, dehydromatricarin, deacetyldehydromatricarin, secotanapartholide A, cyclohexanehexol monomethyl-ethertanaphillin isomer, artanomaloide, arteanomalactone, aurantiamide acetate, anabellamide, trans-o-hydroxycinnamic acid, trans-o-hydroxy-p-methoxycinnamic acid,

Polyporus 25 is the plant of Polyporus umbellatus (Pers.) Fries have containing Polyporusterone A-G, ergosta-4,6,8(14),22-tetraen-3-one, 25-deoxymakisterone A, 25-deoxy-24(28)-dehydromakisterone A, ergosta-7,22-dien-3-one, ergosta-6,22-dien-3-ol, a-hydroxytetracosanoic acid, ergosta-5,7,22-trien-3-ol.

Ramulus Euonymi 26 is the plant of Euonymus alatus Sieb. have containing quercetin, dulcitol, frielin, and resin. Evonoloside, Evozine, Evonine, Evomonoside, Glucoevonoside, β-D-glu-Glucoevonoloside, Evorine.

Flos Sophorae 27 is the plant of Sophora japonica L. have containing azukisaponin I-II, V, soyasaponin I, III, kaikasaponin I-III, quercetin, β-sitosterol, octadecatrienoic acid, myristic acid, rutin, isorhamnetin, isorhamnetin-3-rutinoside, kaempferol-3-rutinoside, betulin, sophoradiol, lauric acid, dodecenoic acid, tetradecenoic acid, tetradecadienoic acid, palmitic acid, hexadecenoic acid, stearic acid, octadecadienoic acid, arachidic acid.

### SUMMARY OF THE INVENTION

The primary purpose of the invention is to disclose some Chinese formula for psoriasis. Whether treatment for psoriasis is targeted at both the hyperproliferative and inflammatory aspect of the topical therapy.

The second purpose of the invention is to disclose the manufacture method of dosage for psoriasis.

### BRIEF DESCRIPTION OF THE DRAWINGS AND FIGS

The invention will now be described by way of example with reference to the accompanying Tables and Figures in which:

Table 1 illustrated antiproliferative activity against HaCaT cells by CTMs.8

Table 2 illustrated inhibitory effects^{a} of various solvents extract of CTMs on LP of rat brain homogenate induced by FeCl₂ in vitro.

Table 3 illustrated prooxidative efects^{a} of various solvents extract of CTMs on LP of rat brain homogenate induced by FeCl₂ in vitro.

### DETAILED DESCRIPTION OF THE INVENTION

The role of oxygen radicals in human disease has become an area of intense interest. There are compilation cause and effect on oxidative tress, lipoproteins and cardiovascular dysfunction etc. Low concentrations of oxygen radicals are constantly formed as physiological byproducts in the human body, but they can be toxic when generated in excess. This toxicity can be of therapeutic interest, depending on the nature of the target cell.

Some of the primary targets of oxygen radicals are the lipids that constitute the cell membrane. Many reactive oxygen species (ROS) are more soluble in a lipid environment than in aqueous systems and can readily cross biological membranes. Although oxygen radicals are responsible for the inflammation of the no affected psoriatic skin, the same species are central to clinical efficacy of CTMs (Chinese traditional medicines). It is common in Western countries, affecting about 2% of the Caucasian population, affecting 1-3% of the American population, but is relatively uncommon in Asia, Lin X R. J. Dermato. 20, 746-55, 1993 have reported the prevalence rate of psoriasis in China was 1.23 0/00 in 1984. The true incidence may be even higher, because individuals with minor clinical manifectations may not seek medical attention, but elect to treat the condition themselves.

The invention elected many of CTMs (Chinese traditional medicines) for psoriasis that including Radix Tripterygiim Wilfordii 1, Caulis Spatholobi 2, Radix Angelicae Sinensis 3, Rhizoma Smilacis Glabrae 4, Rhizoma Sparganii 5, Radix Rehmanniae Exsiccata 6, Radix Ophiopogonis Japonici 7, Radix Asparagi 8, Olibanum 9, Nidus Vaspae 10, Herba Hedyotis 11, Indigo Naturalis 12, Radix Lithospermi 13, Radix Salviae Miltiorrhizae 14, Rhizoma Curcumae Aeruginosae 15, Myrrha 16, Radix Paeoniae Veitchii 17, Pericarpium Citri Reticulatae 18, Flos Carthami 19, Semen Amygdalus Persicae 20, Radix Angelicae Biserratae 21, Radix Angelicae Hangbaizhi, or Radix Angelicae Qibaizhi 22, Rhizoma Imperatae 23, Herba Artemisiae Anomalae 24, Polyporus 25, Ramulus Euonymi 26, Flos Sophorae 27.

Collect some of CTMs together as prescription (A) which is Caulis Spatholobi 2, Radix Rehmanniae Exsiccata 6, Radix Lithospermi 13, Radix Salviae Miltiorrhizae 14, Radix Paeoniae Veitchii 17, Rhizoma Imperatae 23, Flos Sophorae 27. Prescription (B) which is Caulis Spatholobi 2, Radix Angelicae Sinensis 3, Rhizoma Smilacis Glabrae 4, Radix Rehmanniae Exsiccata 6, Radix Ophiopogonis Japonici 7, Radix Asparagi 8, Nidus Vaspae 10, Radix Salviae Miltiorrhizae 14. Prescription (C) which is Caulis Spatholobi 2, Rhizoma Sparganii 5, Herba Hedyotis 11, Rhizoma Curcumae Aeruginosae 15, Pericarpium Citri Reticulatae 18, Flos Carthami 19, Semen Amygdalus Persicae 20, Ramulus Euonymi 26.

To make the solid type of the drugs including tablet, powder, capsule, and granule, the invention of CTMs (Chinese traditional medicines) extracts were added with various additives, such as magnesium stearate, corn powders, starch, lactose, fiber, ethanol, glycerin and so forth, diluent, lubricant, flavouring, disintegrants, binders, coloring agents, sweetener. To make liquid type of the drugs, phosphate buffer solution was added to adjust the pH. This invention was allowed to make the solid or liquid types of the drugs. In addition to the drugs administered by oral or rectal, the injection type or liquid type of effective dosage, non-intestinal injection type, or soft gel type can also be considered for application. Common administration dosage can be specifically prepared according to symptoms, but the usual dosage is 50 mg to 300 mg each time per person, three times per day.

It is generally speaking, administration dosage of the invention are prefect using on soft gel, cream, tincture and aerosol etc. topical dosage. That shall be added with various additives, enhaner such as BHT, Oleth.2 (CTFA), Isoceteth-20 (CTFA), Ascorbyl palmitate, PEG-8 (CTFA), Sorbitol solution, EDTA, Silicon, Sodium bisulfate, Emulage 100 NI, Ascorbic acid, Propylene glycol, Sodium lauryl sulfate. The CTMs extracts usually diluted to expedient concentration, such as 10%, 2%, 5%, then add to mix components of other parts. Those topical dosage of cream or soft gel was prepared by admixing the components of Part A and heating to a melt temperature. Agitation was continued until all of the solids were blended. In a separate vessel, the components of Part B were admixed and heated till melt, with continued agitation until all the solids were dissolved. Part B was mixed into Part A and agitation continued, while maintaining a liquid temperature, until both parts were thoroughly blended. The resulting cream was cooled to packaging temperature and packaged.

The topical dosage of CTMs extracts for psoriasis on this invention, that select from Tripterygiim Wilfordii 1, Caulis Spatholobi 2, Radix Angelicae Sinensis 3, Rhizoma Smilacis Glabrae 4, Rhizoma Sparganii 5, Radix Rehmanniae Exsiccata 6, Radix Ophiopogonis Japonici 7, Radix Asparagi 8, Olibanum 9, Nidus Vaspae 10, Herba Hedyotis 11, Indigo Naturalis 12, Radix Lithospermi 13, Radix Salviae Miltiorrhizae 14, Rhizoma Curcumae. Aeruginosae 15, Myrrha 16, Radix Paeoniae Veitchii 17, Pericarpium Citri Reticulatae 18, Flos Carthami 19, Semen Amygdalus Persicae 20, Radix Angelicae Biserratae 21, Radix Angelicae Hangbaizhi, or Radix Angelicae Qibaizhi 22, Rhizoma Imperatae 23, Herba Artemisiae Anomalae 24, Polyporus 25, Ramulus Euonymi 26, Flos Sophorae 27, prescription (A), Prescription (B), and Prescription (C).

The following H₂O extracts of CTMs are prefect which is Radix Tripterygiim Wilfordii 1, Radix Angelicae Sinensis 3, Radix Ophiopogonis Japonici 7, Olibanum 9, Radix Salviae Miltiorrhizae 14, Rhizoma Curcumae Aeruginosae 15, Myrrha 16, Radix Paeoniae Veitchii 17, Pericarpium Citri Reticulatae 18, Flos Carthami 19, Radix Angelicae Hangbaizhi, or Radix Angelicae Qibaizhi 22, Rhizoma Imperatae 23, Herba Artemisiae Anomalae 24, Ramulus Euonymi 26, Flos Sophorae 27, prescription (A), and Prescription (B). On the other hands, following ethanol extracts of CTMs are prefect which is Rhizoma Sparganii 5, Pericarpium Citri Reticulatae 18, Semen Amygdalus Persicae 20, Herba Artemisiae Anomalae 24, and Polyporus 25.

The CTMs were subjected to be boiled in distilled water at 100° C. for 1 h or organic solvents (ethanol, acetone, dichloromethan) at room temperature for one month, the extract of each CTMs was dried. Then put on Dianon gel, silica gel, and sephadex LH-20 gel column to make the fraction. To understand whether the antipsoriatic CTMs could possess both antioxidative and antiproliferative activity, so the each activity fraction, extract of the 27 CTMs and 3 combination prescriptions be prove through experiments.

On the Dianon gel chromatography column, from 100% H₂O to 100% Methanol of H₂O-Methanol graduate solution are elute to separate the fraction. The graduate solution of Dichloromethan-Methanol, hexane-Ethyl acetate, Ethyl acetate-Methanol, hexane-Dichloromethan graduate solution are elute on silica gel. Graduate solution of H₂O-Methanol, Dichloromethan-Methanol, are elute on sephadex LH-20 gel.

The purpose of the invention was to evaluate the antioxidative properties and antiproliferative activity of CTMs and to determine which CTM is potential treatment for psoriasis. Through the lipid peroxidation of various extract of CTMs and antiproliferative activity against HaCaT cells by CTMs. The antioxidative and antiproliferative mechanisms of these CTMs, however, have not yet been clarified, and it was for this reason that the present investigation was carried out. The results of several CTMs on lipid peroxidation and cytotoxicity against HaCaT cells in vitro enabled us to assess the antipsoriatic activity of CTMs themselves.

Based on the results, the effects of 27 CTMs and 3 prescriptions on lipid peroxidation and antiproliferative activity were evaluated. Some of the investigated CTMs showed significant antioxidation or prooxidation against LP and antiproliferative activity. The IC₅₀ values of antiproliferative activity of CTMs are given in Table 1.

Results from the HaCaT keratinocyte proliferation assay show that most CTMs are significantly efficient, expect 13, 15, 22, 26 and 30. Although all the CTMs showed similar significant inhibition of lipid peroxidation in brain homogenate, some different antioxidative and antiproliferative mechanism could be recognized between them from the following results: (i) in antiproliferative activity assay, some CTMs showed inhibition against HaCaT cells, but some did not; (ii) H₂O-extract of CTMs on LP, only Nos. 1, 2, 4, and 5 showed significant inhibitory effects but Nos. 6, 8, 11, 12, 13, 20, 21 and 30 showed prooxidative activity, respectively; (iii) Ethanol (EtOH)-extract of CTMs on LP, Nos. 1, 2, 4, 6, 7, 8, 9, 10, 11, 13, 14, 15, 16, 22, 26 and 27 showed significant inhibitory effects but Nos. 3, 12, 17, 19, 21 and 23 showed prooxidative activity, respectively; (iv) acetone-extract of CTMs on LP, Nos. 1, 2, 3, 4, 7, 14, 15, 16, 17, 23, 26 and 27 showed significant inhibitory effects but Nos. 9, 13, 19 and 20 showed prooxidative activity, respectively; (v) Dichloromethan-extract of CTMs on LP, Nos. 1, 2, 3, 4, 5, 6, 7, 10, 14, 15, 16, 17, 18, 22, 23, 26 and 27 showed significant inhibitory effects but only Nos. 9, 12, and 13 showed prooxidative activity, respectively. Table 2 shown Inhibitory effects of various solvents extract of CTMs on LP of rat brain homogenate induced by FeCl₂ in vitro. Table 3 shown prooxidative effects of various solvents extract of CTMs on LP of rat brain homogenate induced by FeCl₂ in vitro.

In some cases, the mechanism of antioxidation and antiproliferation in the same CTM differed. For instance, Nos. 13, 15, 22 and 26 showed an inhibition on LP, but this was not observed in antiproliferative activity assay. Nos. 1, 2, 3, 10 and 17 showed an inhibition on LP, and these were also observed in antiproliferative activity assay. In some cases, the results of LP in the various extracts of CTM were also different. For instance, Nos. 3, 6, 8, 9, 11, 12, 13, 17, 19, 20 and 21 showed antioxidative and prooxidative activity, respectively. Nos. 1, 2, 3, 6, 10, 12, 17, 21, 25, 27, 28 and 29 showed significant inhibition in antiproliferative activity assay, but only Nos. 1, 2, 3, 6, 10, 17 and 27 also showed significant inhibition on LP. These results suggest that the inhibition of those CTMs may be an important factor in their biological activity.

A variety of hyperproliferative and inflammatory skin diseases are characterized by increased levels of proinflammatory arachidonic acid metabolites. Reactive oxygen species (ROS) and hydroperoxides that are generated during inflammatory reactions may play an important role in the regulation of these proliferative processes. Lipid is a major target for oxidative damage in cells. Lipid hydroperoxides are key intermediates in the lipid peroxidation process serving as initiators for free radical chain reactions. Because lipid peroxidation can be detrimental to cells, we hypothesized that some CTMs would increase the resistance of cells to oxidative stress. Nevertheless, the relationship between the biosynthesis of lipoxygenase products and ROS is not well defined. The purpose of the invention was to evaluate the effects of CTMs on the lipid peroxidation and antiproliferative HaCaT keratinocyte cells in vitro. The inhibitory effect on lipid peroxidation in brain homogenate and inhibition against HaCaT cells are considered to be due to their peculiar chemical structures and components, as part of their special different chromophore in various extract they scavenge the radicals.

It is well know that CTMs are compound medicines, which are combined with various crude drugs, and the exhibition of their pharmacologic activity is the result of the pharmacodynamic interaction of the combined drugs and their components. Extensive studies have revealed that various CTMs have potential antipsoriatic activity. The structure activity relationship (SAR) of these CTMs is still unclear and requires further research. Up to now, the invention have shown that CTMs can be markedly to a potent treatment for psoriasis. That said that antioxidative and antiproliferative activities of some CTMs strongly correlated with various biological endpoints support the importance of psoriasis. This could be an advantage or disadvantage, depending on the diagnosis and certain disease treatment. The combination use of CTMs is also accompanied by partially diminished oxygen-radical formation and reversal of the enhancement of lipid peroxidation to potent inhibition of psoriatic process. This promises less skin inflammation and suggests an additional protective action against tissue injury. In the future, when treating severe cases with major constitutional disturbances, physician may consider the modality of combining CTMs with modern western medicines in consideration of the patients.

There is no appropriate animal model of psoriasis, for prove the biological activity of drugs useful as antipsoriatic agents even that were evaluated by antiproliferative activity in cell cultures and antioxidative activity.

Table 4 presents the clinical evaluation some psoriasis patients. Comparison of these experimental values for the psoriasis suggests that the effective of the patient for the test compounds is A drug better than B drug.

It is observed in general that a stain of 5 minutes duration can be removed easily with water or solvents. The longer the stain remains on the skin the more difficult it is to remove. This procedure was repeated twice to obtain the best result. Based on effective activity and observed side effect with CTMs is probably due to the result of the therapy process. This was supported by complaint of psoriasis patients in which the major uncomfortable all disappeared. It is therefore reasonable to presume that the intermediate products and stain might be neglect.

Based on the information generated in this study, it is recommended that 2% of extract, whether Radix Tripterygiim Wilfordii 1 is A drug, and Prescription (A) is B drug. The extract used full strength, is the best prescription for psoriasis. For psoriasis patients, the extract should be diluted 1:1,000, 1:5,000 and 1:20,000, respectively. Although the CTMs composition of the present invention are capable of being formulated and used in cream, gel, ointment or tincture, for each of application the cream or tincture forms are prepared. While the present invention has been described by means of the foregoing specification, reference should be had to the appended claim for a definition of the scope of the invention. As a result, several CTMs are currently in preclinical anti-inflammatory, antiproliferative, and toxicological studies.

While the invention is susceptible to various modifications and alternative forms, certain illustrative embodiments thereof have been shown by way of example in the drawing and will herein be described in detail. It should be understood that it is not intended to limit the invention to the particular forms disclosed, but the intention is to cover all modifications, equivalents, and alternatives falling within the spirit and scope of the invention, as defined by the appended claims.

Anyone who is familiar with the said technique is able to amend and/or apply the said technique partially or totally without going beyond the Invention's spirit and coverage. Thus, the protection coverage of the Invention is determined by the descriptions stated in the application of patents.

### PHARMACEUTICAL ACTIVITY

The pharmaceutical activity of the Chinese traditional medicines of this invention has been proven by the following pharmaceutical experiments.

On the basis of this information, evaluated the ability of the Chinese traditional medicines (CTMs) to inhibit the growth of human keratinocytes and lipid peroxidation in model membrane of rat brain homogenate induced by FeCl₂ in vitro.

Chinese traditional medicines (CTMs) used in this study were purchased from market and identified. Thiobarbituric acid, tetramethoxypropane and trichloroacetic acid were purchased from Sigma Chemical Co. Thin layer chromatography (TLC) plastic sheets silica gel 60 F₂₅₄ were from Merck (Darmstadt, Germany). All other materials and solvents were of the highest purity or high-performance liquid chromatography (HPLC) grade. CTMs prepared by their traditional recipe, HaCaT keratinocyte cells were received as a gift from Prof. Dr. K. Müller (Universität Münster, Germany) and Prof. Dr. W. Wiegrebe (Universität Regensburg, Germany).

### HaCaT Keratinocyte Cells Culture

HaCaT Keratinocyte cells were growth in culture using a modification of the method as described by Huang HS. et al., J. Med. Chem. 39:3132-8,1996. In vitro cultured cell system are useful tools in identifying new topical antipsoriatic agents. HaCaT keratinocytes can be used as a model for highly proliferative epidermis, e.g. psoriasis, and this nontransformed human cell line was described as an extremely sensitive target for the antiproliferative action of dithranol (anthralin). by Müller K. Biochem. Pharmacol. 53, 1215-21, 1997. Proliferation of the keratinocytes was determined directly by counting the dispersed cells under a phase-contrast microscope after 48 h of treatment. The CTMs in Table 1 were tested for antiproliferative effects as demonstrated by reduction in cell number over time as compared to control plates.

### Antiproliferative Activity

The immortalized keratinocyte line HaCaT cells were used to mimic the hyperproliferative epidermis found in psoriasis, as antiproliferative action in cell cultures may be critical in the management of the proliferative component of psoriasis. In vitro cultured cell systems are useful tools in identifying new topical antipsoriatic agents. Proliferation of the keratinocyte was determined directly by counting the dispersed cells under a phase-contrast microscope after 48 h of treatment. The Chinese traditional medicines in Table 1 were tested for antiproliferative effects as demonstrated by reduction in cell number over time as compared to control plates. The results of this assay are also provided in Table 1.

### Assay of Lipid Peroxidation

Rat brain homogenate was prepared from the brains of freshly killed Wistar rats and its peroxidation in the presence of iron ions was measured by the thiobarbituric acid (TBA) method as described by Teng CM. et al. Eur J Pharmacol 303:129-39, 1996. Tetramethoxypropane was used as a standard, and the results were expressed as nanomoles of MDA equivalents per milligram of protein of rat brain homogenates. The amount of LP in this method was expressed in terms of malondialdehyde (MDA). In brief, whole brain tissue, excluding the cerebellum, was washed and homogenized in 10 volumes of ice-cold Krebs buffer (10 mM N-2-hydroxyethyl piperazine-N′-2-ethanesulfonic acid (Hepes), 10 mM glucose, 140 mM NaCl, 3.6 mM KCl, 1.5 mM CaCl₂, 1.4 mM KH₂PO₄, 0.7 mM MgSO₄, pH 7.4) using a glass Dounce homogenizer. The homogenate was centrifuged at low speed (1000×g) for 10 min, and the resulting supernatant (adjusted to 2 mg/ml) was used immediately in lipid peroxidation assays. The reaction mixture with test compounds or vehicle was incubated for 10 min, then stimulated by addition of ferrous ion (200 μM, freshly prepared), and maintained at 37° C. for 30 min. The reactions were terminated by adding 10 μl of ice-cold trichloroacetic acid solution (4% (w/v) in 0.3 N HCl) and 200 μl of thiobarbituric acid-reactive substance reagent (0.5% (w/v) thiobarbituric acid in 50% (v/v) acetic acid). After boiling for 15 minutes, the samples were cooled and extracted with 1-butanol. The extent of lipid peroxidation was estimated as thiobarbituric acid-reactive substances and was read at 532 nm in a spectrophotometer (Shimadzu UV-160). The results of this assay are provided in Table 2.

### Clinical Evaluated on Psoriasis Patients

Collect the Clinical psoriasis patients at Trisurvice-general hospital, the 2% cream are contact the 10 mm psoriasis area, 5% are contact the 20 mm psoriasis area, 10% cream are contact over 50 mm psoriasis area.

### EXAMPLE 1

### Preparation of Aqueous CTMs Extract

Powdered of commercially available dried and smashed CTMs (1,000 g) were added with 3,000 ml of distilled water, and boiled until the volume of aqueous extract was reduced to 500 ml. The extracts were pooled and filtered through absorbent cotton. The filtrate was then concentrated under reduced pressure and freeze-dried to give a powder.

### EXAMPLE 2

### Preparation of Organic Layer CTMs Extract

Powered of commercially available dried and smashed CTMs (1,000 g) were added with 2,000 ml of organic solvents (ethanol, acetone, Dichloromethan) at room temperature in the dark room. After one month, the solvent was filtered and concentrated under reduced pressure and freeze-dried to give a brown solid extract powder.

### EXAMPLE 3

### Preparation the Activity Fraction of Aqueous CTMs

Powdered of commercially available dried and smashed CTMs (1,000 g) were added with 3,000 ml of distilled water, and boiled until the volume of aqueous extract was reduced to 500 ml. The extracts were pooled and filtered through absorbent cotton. The filtrate was then concentrated under reduced pressure and freeze-dried to give a powder. Then put on Dianon gel, silica gel, and sephadex gel column to make the activity fraction.

### EXAMPLE 4

### Preparation the Activity Fraction of Organic Layer CTMs

Powered of commercially available dried and smashed CTMs (1,000 g) were added with 2,000 ml of organic solvents (ethanol, acetone, Dichloromethan) at room temperature in the dark room. After one month, the solvent was filtered and concentrated under reduced pressure and freeze-dried to give a brown solid extract powder. Then put on Dianon gel, silica gel, and sephadex gel column to make the activity fraction.

### EXAMPLE 5

### Soft Gel Composition

| Part A | | |
|---|---|---|
| Mineral oil (USP) | 16.0 | g |
| Propyl gallate | 0.01 | g |
| BHT | 0.1 | g |
| Oleth.2 (CTFA) | 6.0 | g |
| Isoceteth-20 (CTFA) | 20.0 | g |
| Ascorbyl palmitate | 0.1 | g |
| CTM extract | 5.0 | g |
| Part B | | |
| PEG-8 (CTFA) | 5.0 | g |
| Sorbitol solution (70%) | 2.0 | g |
| EDTA | 0.01 | g |
| Sodium bisulfate | 0.05 | g |
| Ascorbic acid | 1.0 | g |
| Sodium lauryl sulfate | 0.1 | g |
| Pured water (USP) q.s | 100.0 | |

Admixing the components of part A, and heating to a temperature of 90° C. Agitation was continued until all of the solids were blended. The components of part B were admixed in a separate vessel and heated to 90° C. with continued agitation until all the solids dissolved. Part B was added to part A and the resultant mixture agitated. Agitation was continued for ten minutes, while maintaining the temperature at 90° C. The resulting gel was cooled to packaging temperature and packaged, under an inert gas, in aluminum tubes suitable coated internally so as not to react with the product.

### EXAMPLE 6

### Cream Composition

| Part A | | |
|---|---|---|
| Peanut oil | 4.0 | g |
| Silicon | 3.0 | g |
| BHT | 0.1 | g |
| Prescription (A) extract | 5.0 | g |
| Part B | | |
| Emulage 100 NI | 15.0 | g |
| Sodium phosphate tribasic | 1.0 | g |
| EDTA | 0.1 | g |
| Propylene glycol | 5.0 | g |
| Pured water (USP) q.s | 100.0 | g |

The cream was prepared by admixing the components of Part A and heating to a temperature of 70° C. Agitation was continued until all of the solids were blended. In a separate vessel, the components of Part B were admixed and heated to 70° C., with continued agitation until all the solids were dissolved. Part B was mixed into Part A and agitation continued, while maintaining a temperature of 70° C., until both parts were thoroughly blended. The resulting cream was cooled to packaging temperature and packaged.

TABLE 1
Antiproliferative activity against HaCaT cells by CTMs.

| | | AA^{a} IC₅₀ (□g/ml) |
|---|---|---|
| No | Chinese traditional medicines | H₂O extract |
| 1 | Radix Tripterygiim Wilfordii | 8.1 ± 0.1 |
| 2 | Canlis Spatholobi | 69.7 ± 1.1 |
| 3 | Radix Angelicae Sinensis | 49.5 ± 0.4 |
| 6 | Radix Rehmanniae Exsiccata | 91.7 ± 0.7 |
| 10 | Nidus Vaspae | 84.9 ± 0.8 |
| 12 | Indigo Naturalis | 100.5 ± 1.5 |
| 13 | Radix Lithospermi | 564.2 ± 8.2 |
| 15 | Rhizoma Curcumae Aeruginosae | 467.0 ± 7.1 |
| 17 | Radix Paeoniae Veitchii | 50.9 ± 0.3 |
| 21 | Radix Angelicae Biserratae | 40.9 ± 0.4 |
| 22 | Radix Angelicae Hangbaizhi, or Radix | 424.7 ± 7.5 |
| | Angelicac Qibaizhi | |
| 25 | Polyporus | 54.1 ± 0.6 |
| 26 | Ramulus Euonymi | 425.6 ± 9.1 |
| 27 | Flos Sophorae | 102.5 ± 0.6 |
| 28 | Prescription (A) | 44.1 ± 0.3 |
| 29 | Prescription (B) | 49.9 ± 0.4 |
| 30 | Prescription (C) | 635.1 ± 9.5 |

^{a}Antiproliferative activity against HaCaT cells. Inhibition of cell growth was significantly different with respect to that of the control, N = 3, P < 0.01.
^{b}Prescription (A): 2, 6, 13, 14, 17, 23, and 27.
^{c}Prescription (B): 2, 3, 4, 6, 7, 8, 10 and 14.
^{d}Prescription (C): 2, 5, 11, 15, 18, 19, 20, and 26.

TABLE 2
Inhibitory effects^{a} of various solvents extract of CTMs on LP of rat brain
homogenate induced by FeCl₂ in vitro.

| | | Percentage inhibition of lipid peroxidation^{a} | | | |
|---|---|---|---|---|---|
| | | H₂O | Ethanol | acetone | Dichloromethan |
| No | CTMs | extract | extract | extract | extract |
| 1 | Radix Tripterygiim Wilfordii | 78.9 | 100 | 100 | 100 |
| 2 | Canlis Spatholobi | 100 | 98.5 | 100 | 100 |
| 3 | Radix Angelicae Sinensis | 17.9 | −22.2 | 100 | 100 |
| 4 | Rhizoma Smilacis Glabrae | 97.5 | 98.9 | 100 | 100 |
| 5 | Rhizoma Sparganii | 74.2 | 32.8 | 20.2 | 100 |
| 6 | Radix Rehmanniae Exsiccata | −60.2 | 60.4 | 16.9 | 69.7 |
| 7 | Radix Ophiopogonis Japonici | 8.6 | 87.4 | 100 | 100 |
| 8 | Radix Asparagi | −52.3 | 78.1 | 15.3 | 27.9 |
| 9 | Olibanum | 10.6 | 81.7 | −56.5 | −1.5 |
| 10 | Nidus Vaspae | 55.3 | 94.1 | 26.2 | 100 |
| 11 | Herba Hedyotis | −72.9 | 94.0 | 30.6 | 10.6 |
| 12 | Indigo Naturalis | −70.4 | −51.0 | 13.4 | −3.1 |
| 13 | Radix Lithospermi | −8.7 | 100 | −100 | −100 |
| 14 | Radix Salviae Miltiorrhizae | 9.1 | 100 | 100 | 100 |
| 15 | Rhizoma Curcumae Aeruginosae | 2.4 | 100 | 100 | 100 |
| 16 | Myrrha | 12.9 | 98.0 | 100 | 100 |
| 17 | Radix Paeoniae Veitchii | 19.8 | −27.4 | 100 | 100 |
| 18 | Pericarpium Citri Reticulatae | 0.1 | 20.7 | 35.4 | 100 |
| 19 | Flos Carthami | 15.9 | −24.2 | −19.1 | 48.7 |
| 20 | Semen Amygdalus Persicae | −5.2 | 29.9 | −27.5 | 23.8 |
| 21 | Radix Angelicae Biserratae | −25.2 | −27.7 | 24.1 | 56.2 |
| 22 | Radix Angelicae Hangbaizhi, or | 32.8 | 85.3 | 11.6 | 100 |
| | Radix Angelicae Qibaizhi | | | | |
| 23 | Rhizoma Imperatae | 18.4 | −50.6 | 100 | 100 |
| 24 | Herba Artemisiae Anomalae | 2.9 | 31.1 | 10.3 | 45.9 |
| 25 | Polyporus | ND^{b} | 26.3 | ND^{b} | ND^{b} |
| 26 | Ramulus Euonymi | 16.4 | 100 | 100 | 100 |
| 27 | Flos Sophorae | 0.6 | 92 | 100 | 66.5 |
| 28 | Prescription (A) | 27.4 | ND^{b} | ND^{b} | ND^{b} |
| 29 | Prescription (B) | 32.0 | ND^{b} | ND^{b} | ND^{b} |
| 30 | Prescription (C) | −3.8 | ND^{b} | ND^{b} | ND^{b} |

^{a}Relative percentage of inhibition, P < 0.01, Mean ± S.E., n = 4. Values in parentheses are percent inhibition at the indicated concentration (100 □g/ml), and standard errors average 10% of the indicated values.
^{b}ND = not determined.

TABLE 3
Prooxidative effects^{a} of various solvents extract of CTMs on LP of rat
brain homogenate induced by FeCl₂ in vitro.

| | | Percentage prooxidation of LP^{a} | | | |
|---|---|---|---|---|---|
| | | H₂O | Ethanol | acetone | Dichloromethan |
| No | CTMs | extract | extract | extract | extract |
| 6 | Radix Rehmanniae | −60.2 | | | |
| | Exsiccata | | | | |
| 8 | Radix Asparagi | −52.3 | | | |
| 9 | Olibanum | | | −56.5 | −1.5 |
| 11 | Herba Hedyotis | −72.9 | | | |
| 12 | Indigo Naturalis | −70.4 | −51.0 | | −3.1 |
| 13 | Radix Lithospermi | −8.7 | | −100 | −100 |
| 19 | Flos Carthami | | −24.2 | −19.1 | |
| 20 | Semen Persicae Persicae | −5.2 | | −27.5 | |
| 23 | Rhizoma Imperatae | | −50.6 | | |

^{a}Relative percentage of inhibition, P < 0.01, Mean ± S.E., n = 4. Values in parentheses are percent inhibition at the indicated concentration (100 □g/ml), and standard errors average 10% of the indicated values.

TABLE 4
Clinical evaluated on psoriasis patients

| | Administration | Clinical | response No. of patients/ |
|---|---|---|---|
| drug | time | response | total No. of patients |
| 0.1% A drug | 30 days | excellent | 7/12 |
| | | better | 4/12 |
| 0.5% A drug | 30 days | excellent | 8/12 |
| | | better | 3/12 |
| 2%A drug | 30 days | excellent | 9/12 |
| | | better | 2/12 |
| 0.1% B drug | 30 days | excellent | 4/12 |
| | | better | 3/12 |
| 0.5% B drug | 30 days | excellent | 5/12 |
| | | better | 3/12 |
| 2% B drug | 30 days | good | 6/12 |
| | | better | 2/12 |

A drug: Radix Tripterygiim Wilfordii 1 A drug
B drug: Prescription (A)

We claim:

1. An topical dosage of antipsoriatic Chinese traditional medicines whether select from one or more then one of Radix Tripterygiim Wilfordii 1, Radix Angelicae Sinensis 3, Radix Ophiopogonis Japonici 7, Olibanum 9, Radix Salviae Miltiorrhizae 14, Rhizoma Curcumae Aeruginosae 15, Myrrha 16, Radix Paeoniae Veitchii 17, Pericarpium Citri Reticulatae 18, Flos Carthami 19, Radix Angelicae Hangbaizhi, or Radix Angelicae Qibaizhi 22, Rhizoma Imperatac 23, Herba Artemisiae Anomalae 24, Ramulus Euonymi 26, Flos Sophorae 27, prescription (A), and Prescription (B).

2. The topical dosage of antipsoriatic Chinese traditional medicines according claim 1, whether administration dosage select from soft gel, cream, tincture and aerosol.
